(19) United States
(12) Patent Application Publication
(10) Pub. No.: US 2014/0273138 A1
(43) Pub. Date: Sep. 18, 2014

(54) Simultaneous Food And Fuel Corn Refining

(71) Applicant: Leon H. Langhauser, Decatur, IL (US)

(72) Inventor: Leon H. Langhauser, Decatur, IL (US)

(21) Appl. No.: 14/203,910

(22) Filed: Mar. 11, 2014

Related U.S. Application Data
(60) Provisional application No. 61/776,865, filed on Mar. 12, 2013.

(51) Int. Cl. C12P 7/10; A23K 1/00

(52) U.S. Cl. CPC C12P 7/10; A23K 1/007
USPC 435/165; 426/18; 426/28

(57) ABSTRACT

Food grade products are given priority with a sustainable integrated corn based bio grind refining process. Premium fractions are fractionated for human foods and premium fermentable products. The balance food feedstocks are refined for animal feed with no loss of nutritional value. By-products of refining are used to produce ethanol and other energy products. There is no process waste. The integrated processes can be adapted to new continuous refineries or to optimize or retrofit one or more individual process steps.

### CROSS-REFERENCE TO RELATED APPLICATION

This application claims the benefit of U.S. Provisional Application Ser. No. 61/776,865, Mar. 12, 2013.

### FIELD OF THE INVENTION

This invention relates to chemistry. More particularly, this invention relates to the food processing. Still more particularly, this invention relates to the refining of corn and other plant materials.

### BACKGROUND OF THE INVENTION

1. Corn

A variety of cereal grains and other plants are grown for use as food. Major cereal grains include corn, rice, wheat, barley, sorghum (milo), millets, oats, and rye. Other plants include potatoes, cassaya, and artichokes. Corn is the most important cereal grain grown in the United States. Corn is sometimes called maize and has the scientific name Zea mays. A mature corn plant consists of a stalk with an ear of corn encased within a husk. The ear of corn consists of about 800 kernels on a cylindrical cob. The kernels are eaten whole and are also processed into a wide variety of food and industrial products. The other parts of the corn plant (i.e., the stalk, leaves, husk, and cob) are commonly used for animal feed, but are sometimes processed into a variety of food and industrial products.

In more detail, the corn kernel consist of three main parts: (1) the pericarp; (2) the endosperm; and (3) the germ. The pericarp (also known as the seed coat or bran) is the outer covering of the kernel. It consists primarily of relatively coarse fiber. The endosperm is the energy reserve for the plant. It consists primarily of starch, protein (also known as gluten), and small amounts of relatively fine fiber. The germ (also known as the embryo) consists primarily of oil and a miniature plant with a root-like portion and several embryonic leaves.

2. Starch

Starch is stored in a corn kernel in the form of discrete crystalline bodies known as granules. On a molecular level, starch is a polymer of anhydroglucose units (C₆H₁₀O₅). Anhydroglucose units combine with a water molecule (H₂O) to produce the common sugar glucose (C₆H₁₂O₆) so readily that starch is commonly referred to as a polymer of glucose. Starch is a member of the general class of carbohydrates known as polysaccharides. Polysaccharides contain multiple saccharide units (in contrast to disaccharides which contain two saccharide units and monosaccharides which contain a single saccharide unit). Polysaccharides made up of the same saccharide units (such as cellulose) are sometimes referred to as homopolysaccharides while polysaccharides made up of different saccharide units are sometimes referred to as heteropolysaccharides.

The length of a saccharide chain (the number of saccharide units in it) is sometimes described by stating its “degree of polymerization” (abbreviated to D.P.). Starch has a D.P. of 1000 or more. Maltose is a disaccharide (its D.P. is 2) that is composed of two glucose units. Glucose (also known as dextrose) is a monosaccharide (its D.P. is 1).

Saccharides having a D.P. of about 5 or less are sometimes referred to as sugars. Monosaccharide sugars containing six carbon atoms (e.g., glucose) are sometimes referred to as hexoses and sugars containing five carbon atoms are sometimes referred to as pentoses.

The anhydroglucose units in starch are connected to each other in one of two ways. When connected together in alpha-1,4-linkages, the starch molecule is linear. When connected together in alpha-1,6-linkages, a branch occurs. The relative number of the two linkages varies depending on the variety of corn. Both types of linkages are sometimes referred to as glucosidic linkages.

3. Fiber

As mentioned above, the pericarp and endosperm of the corn kernel contain fiber. The fiber comprises cellulose, hemicellulose, lignin, pectin, and relatively small amounts of other materials. Fiber is present in relatively small amounts in the corn kernel, but is present in much greater amounts in other corn components such as the cob, husk, leaves, and stalk. Fiber is also present in other plants. The combination of cellulose and lignin is sometimes known as lignocellulose and the combination of cellulose, lignin, and hemicellulose is sometimes known as lignocellulosic biomass. As used herein, the term “fiber” (and its alternative spelling “fibre”) refers to cellulose, hemicellulose, lignin, and pectin. Each of the components of fiber is discussed in detail below.

Cellulose, like starch, is a polymer of anhydroglucose units (C₆H₁₀O₅). However, where the anhydroglucose units in starch are connected to each other in alpha-1,4 and alpha 1,6-linkages, the anhydroglucose units in cellulose are connected to each other in beta-1,4-linkages which give the cellulose molecule a linear, chain-like configuration. Cellulose is a rigid, crystalline structure because its molecules form attractions, known as hydrogen bonds, with adjoining molecules. Cellulose can be converted to glucose by breaking the beta-1,4-linkages by treatment with enzymes and/or by treatment at high temperatures and pressures in the presence of water.

The beta-1,4-linkages in cellulose are not broken down in the human digestive system. Accordingly, cellulose provides no nutritional benefit to humans and passes through the digestive system intact. Cellulose in the human diet is often referred to as fiber or roughage. In contrast to humans, some mammals are able to digest cellulose. Ruminant animals, such as cattle, sheep, goats, and deer, have certain types of bacteria in their digestive systems that produce enzymes that can break down the beta-1,4-linkages to free individual glucose molecules.

Hemicellulose is a heteropolysaccharide that, like cellulose, is present in the corn kernel and in the cell walls of other plants. On a molecular level, hemicellulose is a polymer of several pentose and hexose sugars, including xylose, mannose, galactose, arabinose, and glucose. Where cellulose molecules are linear and form a rigid crystalline structure, hemicellulose molecules are branched and form a much weaker structure.

Lignin is a complex compound composed of linked six-carbon phenolic rings that is present in the corn kernel and in the cell walls of other plants. After cellulose, lignin is the most abundant organic molecule on Earth. Lignin is a non-crystalline substance that acts as a binder of the cellulose in plants.

Pectin is heteropolysaccharide that is also present in cell walls. On a molecular level, pectin is a polymer of several compounds, including galacturonic acid, rhamnose, galactose, arabinose, and xylose.

4. Conventional Corn Refining Processes

A wide variety of processes have been used to separate the various components of corn. These separation processes are commonly known as corn refining. One of the processes is known as the dry milling process. In this process, the corn kernels are first cleaned and then soaked in water to increase their moisture content. The softened corn kernels are then ground in coarse mills to break the kernel into three basic types of pieces—pericarp, germ, and endosperm. The pieces are then screened to separate the relatively small pericarp and germ from the relatively large endosperm. The pericarp and the germ are then separated from each other. The germs are then dried and the oil is removed. The remaining germ is typically used for animal feed. The endosperm (containing most of the starch and protein from the kernel) is further processed in various ways. As described below, one of the ways is to convert the starch to glucose and then ferment the glucose to ethanol.

A second corn refining process is known as the wet milling process. In this process, the corn kernels are first cleaned and then steeped (soaked) in warm water containing sulfurous acid (H₂SO₃). During steeping, water soluble proteins and other substances dissolve into the steep water. After steeping, the softened corn kernels are ground in coarse mills to break the kernel without damaging the germ. The kernels then flow to centrifugal separators which separate the less dense germs from the denser pericarp and endosperm. The germs are then dried and the oil is removed.

The pericarp and endosperm are then ground in fine mills. The finely ground stream flows to screens which separate the small particle size pericarp from the larger particle size endosperm. The endosperm stream then flows to centrifugal separators that separate the less dense protein from the denser starch. The finished starch is in granular form and is suitable for many different types of further processing.

For example, the starch can be dried and sold as unmodified starch. The starch can be modified and used for food or industrial purposes. The starch polymer can be partially hydrolyzed (i.e., shortened or reduced in D.P.) to produce corn syrup or hydrolyzed all the way to the individual glucose units. If completely hydrolyzed to glucose, the glucose molecules can be isomerized to fructose. Fructose is considerably sweeter than glucose and is widely used in the food industry. The starch can also be used for fermentation, as described in more detail below.

5. Fermentation Processes

Fermentation is a process by which microorganisms such as yeast digest sugars to produce ethanol and carbon dioxide. The basic reaction is

C₆H₁₂O₆→2C₂H₅OH+2CO₂

Yeast reproduce aerobically (oxygen is required) but can conduct fermentation anaerobically (without oxygen). The fermented mixture (commonly known as the beer mash) is then distilled to recover the ethanol. Distillation is a process in which a liquid mixture is heated to vaporize the components having the highest vapor pressures (lowest boiling points). The vapors are then condensed to produce a liquid that is enriched in the more volatile compounds.

Various processes have been disclosed for producing fuel ethanol from corn. The processes all convert the starch present in the corn kernel into glucose which is then fermented. For example, one process uses the endosperm isolated from a dry milling process as the feed material. This process is illustrated in Figure One. Another process uses the starch isolated from the wet milling process as its feed material. This process is illustrated in Figure Two. Conventional processes for producing fuel ethanol from corn produce a maximum of about 2.6 to 2.8 gallons of fuel ethanol per bushel of corn. Conventional processes do not convert any of the lignocellulose materials in corn to sugars and, therefore, the lignocellulose does not contribute to ethanol production.

6. The Langhauser Processes

Two efficient processes for producing ethanol from corn are disclosed in Langhauser, U.S. Pat. No. 7,452,425, Nov. 18, 2008 (“Langhauser I”), and Langhauser, U.S. Pat. No. 7,488,390, Feb. 10, 2009 (“Langhauser II”), both of which are hereby incorporated by reference in their entireties. The Langhauser processes are sometimes referred to collectively herein as the Langhauser Associates, Inc. (“LAI”) processes.

The Langhauser I process is illustrated in Figure Three. The Langhauser II process is illustrated in Figure Four. The Langhauser I process produces a coarse fiber stream (consisting of cellulose from the pericarp and various other pericarp components including hemicellulose, lignin, pectin, and sugars) as a co-product. The Langhauser II process converts most of the fiber to glucose. The glucose is then fermented with glucose from starch.

7. Current Economic Conditions

As the world population and demand for fuels both increase, there is an increased demand for both food and ethanol from corn. Approximately one-third of the corn grown in the United States is refined into ethanol. The price of corn reached record high levels in 2012 due to drought and other factors. The high price of corn made the conversion of corn to ethanol economically unattractive and many ethanol manufacturing plants closed. Accordingly, there is a demand for an improved process that more economically refines corn into ethanol.

### SUMMARY OF THE INVENTION

The general object of this invention is to provide an improved process for refining a plant material such as corn. A more particular object is to provide an improved process for refining corn into ethanol. Another more particular object is to provide an integrated corn based bio grind refining (ICBR) process for refining corn and other fiber-containing materials into ethanol.

I have invented a sustainable process for producing ethanol from corn, cereal grain, adjuncts of cereal grain processing, corn stover, adjuncts of biomass material, starch containing tubers, or starch containing adjuncts of rejects or re-process materials. The process combines five processes: (a) corn wet grind with food fractionation with continuous steeping; (b) corn dry grind with cereal grin adjunct and counter flow bi-products and wash water blending; and (c) corn bio grind with LAI-Supramyl high solids feed, high speed centrifugal colloid mill, combination fine grind/liquefaction with high shear and low viscosity discharge; (d) cellulose grind; and (e) combined fermentation.

The discharges are separated for addition to the combined fermentation system and the animal feed refining system with the balance to feed the cellulose grind. The cellulose grind utilizes available fiber, heat, water, catalyst, chemicals, enzyme and yeast recycles from the bio grind, adjunct from the dry grind and additional feedstock material containing cellulose, hemicellulose, lignin, and pectin.

The combined fermentation process includes three continuous fermenters comprising: (i) The bio grind continuous cascade fermenter process with carbon dioxide fine bubble supplemental mixing and blanketing for anaerobic yeast ethanol production; (ii) a one stage continuous pentose utilizing recycled catalyst and pervaporation filter fractionation of ethanol discharged to the primary fermenter for concentration; and (iii) a one stage fermenter for the discharge of the cellulose/dextrose hydrolysate process. The evaporation and animal feed product is combined with the bio grind fermentation and animal feed processes. The animal feed fraction is washed to the fermenters. The fiber, corn steep syrup and protein are blended with a priority to produce the corn value products with the best demand and price. There is no process product loss or waste of nutritional value.

### BRIEF DESCRIPTION OF THE DRAWINGS

Figure One is a schematic diagram of a prior art dry grind corn refining process for the production of fuel ethanol.

Figure Two is a schematic diagram of a prior art wet grind corn refining process.

Figure Three is a schematic diagram of a prior art bio grind corn ethanol refining process disclosed in Langhauser I.

Figure Four is a schematic diagram of a prior art bio grind process with a fermenter discharge and lignocellulosic feedstock bolt-on disclosed in Langhauser II.

Figure Five is a schematic diagram of step one of the ICBR food priority process of this invention.

Figure Six is schematic diagram of step two thereof.

Figure Seven is a schematic diagram of step three thereof.

Figure Eight is a schematic diagram of step four thereof.

Figure Nine is a schematic diagram of step five thereof.

Figure Ten is a schematic diagram of step six thereof.

Figure Eleven is a schematic diagram of step seven thereof.

Figure Twelve is a schematic diagram of the complete ICBR process.

Figure Thirteen is a first chart that compares the ICBR process of this invention with prior art processes.

Figure Fourteen is a second chart that compares the ICBR process of this invention with prior art processes.

### DETAILED DESCRIPTION OF THE INVENTION

1. The Invention In General

The Langhauser III integrated corn based bio grind refining (ICBR) process of this invention relates to the integration of established Dry Grind, Wet Grind, Bio Grind and Bio Mass Grind processes for the fractionation of Corn Steep Solids (CSS), Germ, Gluten, Starch and Fiber for quality feedstocks for refining pharmaceuticals, food products, chemicals, industrial products, and animal feeds with the balance going to ethanol and other energy products. Bio Mass Grind by-products not used for animal feed can be used for boiler feedstock, gasification, animal bedding or compost. The process retains food nutrition value while producing increased ethanol yields, quality animal feeds and added energy value with reduced water and energy and no process waste.

The invention is a sustainable method of continuous refining of food products of Corn Oil, Corn Starch, Gluten Protein and Dietary Fibers with priority fractionation processes utilizing established Wet Grind Procedures. The balance of the stream including wash waters and refining by-products can be diverted to the BioGrind Ethanol Refinery.

The process can be installed in seven steps illustrated in Figures Five through Eleven. Each step offers technology and improvements self-stranding or as part of the complete ICBR process illustrated in Figure Twelve. The steps can be sized to be fabricated, skid mounted, shipped, installed, and operated in any remote area of the world at 2,000 to 6,000 bushels of corn or corn equivalent per day. The process can be utilized for economy of size related to furnishing capacity planned for products. The plant can be at one location or at a number of nearby plants for economy of shuttling product and storage of feedstocks.

Cleaned 15-30% moisture corn is steeped and washed with or without sulfur dioxide using LAI Continuous Steeping Technology utilizing anaerobic processing to avoid 3-7% fermentation sugar losses and membrane technology to reduce steeping time and increased corn syrup solids (CSS). The corn moisture is raised to 44-46% moisture for course grinding to free the germ for fractionation. The CSS is used for fermentation catalyst. Both products are food quality.

The germ is ground and hydraulically processed to remove soil, sand and similar contaminants and the overflow (O/F) of germ at 50% dry substance (DS) oil and 50% DS meal with the under flow (U/F) the balance of the grain product. After the germ is fractionated, the germ can be dried for storage or conditioned for reefing Crude Corn Oil, Degummed Corn Oil or Refined Corn Oil. The balance of the O/F can be finished to a quality poultry feed—Corn Oil Meal. The U/F can be directed to the fine grind for Feedstock fermentation and/or refining pharmaceuticals, specialty foods, fine chemicals, bio chemicals and industrial products. The U/F can also be fractionation of for starch, protein and fiber food products or process and refined to Starch based foods with the balance used to blend with Dry Grind course ground product of grain and tuber adjunct to adjust the feedstock for Bio Grind Supramyl liquefaction.

The LAI-Supramyl process is capable of unlimited shear, 42% solids and processing with reduced viscosity with elimination of the typical viscosity peak internally for a reduced viscosity discharge. The process is the lowest energy consumption, the lowest chemical usage, the highest solids level, the smallest footprint, the lowest steam pressure at 5 bar which adds up to a 5% ethanol yield over most of the dry grind liquefaction processes available.

The liquefied starches are cooled and pH adjusted and enzyme treated to lower the viscosity and raise the dextrose equivalent to 50-60 for efficient fractioning and washing of the fiber and gluten insoluble solids. The underflow of the four stage washing process is directed as feedstock of the LAI Continuous Cascade anaerobic fermentation process. The overflow of the fractionation process is washed and dewatered to be used for Cellulose Grind feedstock or blended with recycled CSS for blending to produce Wet or Dry Distillers Grains with Solubles (DGS), Gluten meal, Gluten Feed. The products can be low starch, low oil or high protein as desired for specialty animal rations. The WDGS product before blending becomes a conditioned feedstock for the LAI Cellulose Grind at half the cost of collecting, storing and conditioning corn stover or other starch containing biomass.

Yeast is propagate with air control to limit the usage of sugar for ethanol yield. Corn sugar sweet wash water is an excellent feedstock for yeast propagation. The LAI fermentation systems are continuous and designed to operate as both batch or continuous for maintenance as needed. The agitation is by mechanical and supplemental carbon dioxide gas fine bubble mixing. The system is blanketed with the gas carbon dioxide to insure anaerobic fermentation, and washing, collecting, refining and liquefying the gas. The gas is used for pH control and cooling the product as well as marketed to the food and beverage industry. This is important for the control of contaminants contributing to yield loss and catalyst recycle quality feedstock for cellulose fermentation that discharges into the primary fermentation system for concentration and yields of ethanol feeding the steam distillation process.

To take advantage of the bolt-on economics of the Cellulose Grind, is to provide additional cellulose adjunct to utilize available recycle catalyst, heat, water and fermentation and distillation capacity without major additions. The entire ICBR process with cellulose bolt on will improve yield of ethanol 5% over Dry Grind plants, 8.5% over Wet Grind Food plants and an increase of 15% due to the addition of the Cellulose Grind. For a Gallon per Dent corn bushel equivalent of four (4) gallons per bushel. The entire system will have controlled environmental discharge with all air discharges with waste caustic pH controlled scrubbers to control PM10, odors, process waste for re-use recycle and solids not used for animal feed gasified or used for boiler feedstock, cattle bedding or land mulch.

To take advantage of the economics of size to utilize standard proven equipment, a minimum of 300,000 bushel of Dent Corn, 60,000 bushels of corn stover equivalent for Dry Grind adjunct and 750 ton equivalent corn cob biomass for Cellulose Grind is made available for fractionating and processing at one or more locations. The primary Dent Corn feedstock will be steeped for the fractionation of Food Grade Germ, with the balance of the grain available for quality food value added fermentation or commercial processing. Fractionated germ is fractionated for oil and Corn Oil and Corn Oil Meal at one central location and Fractionation of Food starch, Gluten and Dietary Fiber for refining will be at a location that can direct the mash after the steep water syrup and the germ has been fractionated which contains 2000 tones of starch or corn equivalent of 120,000 bushels per day.

The 300,000 bushels of dent corn would be used for continuous steeping of dent corn with or without cleaning, with or without sulfur dioxide addition for fractionating soluble proteins utilizing 15-30% moisture corn, anaerobic membrane processing procedures to control wild yeast fermentation and contaminant growth, improve water up-take time and wash the corn for course grinding and fractionating the germ with a standard hydrocylone system. The germ can be conditioned with cold starch liquefaction enzyme, washed and conditioned for oil fractionation, pressing, extraction and refining as desired.

The Dry Grind feedstock should be the corn starch equivalent of corn or other readily available adjunct of 60,000 bushel per day. The grain is course ground and combined with the underflow and food refining byproducts from the germ fractionating underflow stream. The LAI Bio Grind Suproamyl Liquefaction Process can handle Dry Substance at 40-42% Dry substance with the reduced viscosity control.

The BioGrind feedstock is simultaneously liquefied and fine ground utilizing the Supraton rotary stator liquefaction process which requires less enzyme lower temperature, lower steam pressures, less time and reduces the viscosity at higher solids for more shear and conversion efficiency to dextrose due to the shattering of the starch granule.

The Supraton discharge is cooled, pH adjusted and treated with Saccharification enzymes to control flocculation and retrogradation products of multulose and isomaltose to further reduce viscosity and improve ethanol yield and process operations. The insoluble Gluten Protein and fiber is fractionated to provide a clean feedstock to the continuous fermenter system to provide ample dextrose to exceed 18-20 fermenter ethanol, add yeast and catalyst and still have room to concentrate pervaporation concentrate from the lignocellulosic pentose and dextrose single stage fermenter reactors.

The cellulose stream is subjected to a mild alkaline treatment, enzyme hydrolysis and pentose fermentation. Catalyst utilized for the Pentose fermentation will typically be re-use catalyst from the dextrose primary fermentation which contains pentose sugars not utilized by the yeast. The mild alkaline treatment will be followed by a severe alkaline treatment for cellulose starch enzyme hydrolysis and dextrose fermentation. Ethanol is fermented from both fermenter processes and fractionated by pervaporation-distillation and concentrated along with the Bio Grind continuous fermenter which feeds the grain distillation.

The underflow of the insoluble gluten protein and fiber overflow fractionation which has been treated for saccharification of dextrose is combined with steep water solids catalyst, pH adjusted and cooled for fermentation feedstock and combined with propagated yeast for continuous cascade fermentation. The fermenters can be operated batch or continuous. The fermenter system is fitted with fine bubble carbon dioxide mechanical agitation assist and blanketing to control for anaerobic fermentation to improve yield and retard contaminant growth.

The byproducts of all the processes are combined to produce Wet and Dry Distillers Grains w/Syrups, Corn Gluten Meal, Corn Gluten Feed, boiler feedstocks and gasifiers as needed.

The process of this invention refines corn, other cereal grains, corn stover and other plant materials with the potential to add adjuncts of starch and cellulose. The process of this invention is a reduction in energy and costs and an improvement in Quality, Yield of Ethanol along with fractionation of value added products of food grade products from starch, gluten and fiber, retaining of nutritional value and recycles of chemicals, water, steam and process refining solids for no process wastes. Food, Feed, Fiber and Fuel is processed by the integration of the Wet, Dry, Bio and Cellulose Grinds for a sustainable corn and cellulose continuous refinery for a combined 72% starch yield with a three (3) gallon per bushel equivalent Fuel Ethanol yield and with an increase of 25% cellulose added yield utilizing the recycles and remaining fiber after the fractionations of Food and Animal feed are complete. A preferred embodiment of the overall process is illustrated in simplified form in Figure Twelve. The integrated process produces Fuel Ethanol in yields significantly greater than those achieved in prior art grinds both independent and of plurality of two or more of the four processes

2. Grinding Capacity

The preferred capacity for the sustainable integrated process is 300,000 to 500,000 bushels per day of corn equivalent to provide sufficient fractionated product to use existing available equipment to economically refine the corn based products. The grind could be in one location or in a number of close by smaller plants. Costs to integrate the processes would be significantly reduced if the plants would have existing corn grind process that could be retrofitted for the integrated grind of this invention. Pretreatment, processing, and fractionation can take place at all of the locations and resulting fractions can be transported to a central location at a preferred refining capacity. This novel distribution process reduces feedstock delivery cost, transportation backhaul costs of fuel ethanol producing smaller retrofitted plants, increases ethanol throughput and improves the overall economics of ethanol production.

3. Collection, Conditioning And Storing

The preferred feedstock corn and corn stover can be handled and processed directly from the field without process changes at a wide range of moistures of 10% to 30+%. All feedstock can be handled at these moistures except typical hammer mill/screen grinders used for Dry Grind fine grind which requires a 16% moisture. The Dry Grind utilizes a course grind for the integrated grind and can retrofit with a Stedman pin mill (no screens) for the higher moistures. The stover can be windrowed and field chopped and delivered for processing directly to the processing plant with a short time storage without drying. Adjunct grasses and other stovers can be handled in a similar fashion directly from the growing fields using existing design harvesting equipment.

If the grain requires storage, it can be handled with the same conditioning, drying, shipping and storage equipment. Grain is typically dried with commercial dryers. The stovers and grasses typically are windrowed directly from the harvester and conditioned, dried and increased in density with the same equipment used for field chopping and baling as used for harvesting roughage for cows and other ruminants. Chopped and baled can be added directly to the continuous conditioning process using equipment currently used to condition baled cotton lint for mild alkaline or acid treatment. This step is illustrated in schematic form in Figure Five.

4. Wet Grind Soaking, Steeping and Washing

The next step with the ICBR process is to soak and steep the corn to fractionate the Corn Steep Liquor (CSL) for use as catalyst for ICBR fermentation processes or concentrated for marketing. The preferred steeping process is the Langhauser Continuous Steep as illustrated in schematic form in Figure Six. The corn can be cleaned or used as delivered with scalping only and with sand removal after steeping, The corn is distributed on the top of the steep above the recycle CSL. The corn is steeped with water from downstream recycle wash water with or without sulfur dioxide added and used to wash the steeped corn before discharge. The corn is packed for plug flow down the steep before being expanded for washing and discharge from the 57 degree cone with no internals in the steep. The wash water is de-aerated as is the recycle CSL for anaerobic steeping to control contaminants and retard aerobic fermentation by wild yeasts and bacteria for a starch yield improvement of 3-5% as the retained sugars become feedstock for the ethanol fermenters via the starch or the CSL. The continuous steep is preferred to be 70 or more feet to provide internal tank pressure to provide conditions of for the membrane transfer of solubles through the outer corn fiber course fiber membrane for reduced steeping times and improved CSL solids and yield. The recycle is added at the top of the cone for counter flow water flow and expanding to wash and discharge. The water is removed below the dry corn level using a self cleaning screen. The germ moisture at discharge is 44-46% moisture and the CSL is at 6.8-7.0% DS.

5. Wet Grind Fractionation

The steeped and washed corn is discharged from the steep at 44-45% moisture for the next step to coarsely grind the kernels to break them apart for fractionation of the Food Grade Corn Germ which is separated into Corn Oil and Corn Oil Meal (COM), Corn starch which is used for Corn Starch refining and starch based products and starch based refining feedstock including some soluble starches an chemicals, Liquefied for sugar products and sugar based refining feedstock, course fiber for separating fiber starch, Corn Grits, Gluten fiber for corn gluten products and gluten based feedstock based products and Fine fiber for dietary fiber and other fiber products. A preferred course grinding step is illustrated in Figure Six in which the steeped kernels are ground sequentially in two course grinding mills. A preferred course grinding mill is a cage mill containing cage pins and breaker plates a commercial product of Stedman Machine Company of Aurora, Ind. or a disc refiner, a product of Andritz of Muncy, Pa. The pliable germ is slurried with the ground grain at 17-18% Dry Substance for easy removal by hydroclone fractionation. The fractionated food grade product is further refined to Corn Oil and Corn Oil Meal which is a premium poultry supplement.

6. Intermediate Grinding

One of the major reasons the integrated process provides sustainability is the reduction of foot print and equipment needs of the continuous process along with the high construction and operating costs associated with the Food Grade Wet Grind due to the refining required to provide starch yield and separate the starch and gluten protein. With the integrated process the production of Fine Fiber is limited and yield of starch and gluten protein yield is not important. Fine screens and high speed disc centrifuges for separation and concentration are not necessary as only the products needed are fractionated and refined with all of the balance and co-products not needed can be diverted as feedstock can used with or without grinding to operate the Dry, Bio, and Cellulose Grinds which utilizes fine grinding as part of the Supramyl Liquefaction process. Typically, a Double Disc Refiner provided by Andritz of Muncy, Pa. is used for the intermediate grind. The course fiber is fractionated before grinding for providing feedstock to process Fiber Oil as needed with the balance going to the dry grind process. The discharge of the grinder is screened for fractionating Corn Grits for processing and drying. The balance is used to wash and fractionate food starch, gluten protein and dietary fiber as needed with the balance diverted to the dry grind for blending with the product for Bio Grind feedstock. The fractionated products are used as Food or processed feedstock for Fermentation, Fine Chemical, bio Chemicals, pharmaceutical sugars, protein processes or diverted for animal feeds. There is no process product losses.

7. Dry Grinding

Typical handling of the grain and adjunct can be used to grind the products for soaking. The grind can handle higher moistures in the 16-18% range because the final grinding will be with the LAI Supraton Liquefactions process. Wash water from the Food wet grind is used to soak and continuous adjust the control and solids for feeding the bio grind. There is no need for additional fresh water to operate water scrubbers for PM10, dust and odor control and all starch dust is reclaimed for ethanol production and animal feed yield. Only the first step of Figure One of the dry grind process is used because the ground feedstock is blended with the discharge of the wet grind and becomes the feedstock of the bio grind.

8. Liquefaction with Simultaneous Fine Grind and Saccharification

Another major reason the Integrated Corn Based Refining (ICBR) process provides sustainability is the increase of solids levels up to 42% DS from 36% due to the internal reduction of viscosity of the LAI Supramyl process. The ICBR Fine Grind Liquefaction is illustrated in Figure Eight which utilizes the feed from the ICBR Dry Grind. Less chemicals, temperature and recycle steam for better quality and retention of nutritional value of animal feed co-products. The process uses less water and requires less evaporation for increased energy reduction and operating cost savings. The liquefaction is preferably performed in a suitable rotary homogenizer. The slurry is pumped under pressure into a chamber and is forced laterally. The result is a pulsing flow with a rapid succession of compression and depression producing a shattering/shear of the starch granule increasing surface for enzyme reaction.

The process provide a starch yield and sugar conversion that produces a yield of three (3) gallon of Fuel Ethanol per corn equivalent bushel of corn. The sugar is washed and fractionated from the animal feed co-products as feedstock for the sugar conversion primary Fermenter. Fermentation is preferably conducted in a continuous cascade integrated reactor as illustrated in Figure Nine. The fermenter is operated with all the advantages of a clean feedstock continuous anaerobic system. When retrofitted from a Dry Grind full mash feedstock fermenter system there will be from 10-20% equipment available to be used for the ICBR yield increases. The balance of the animal feed products not used for animal feed fractionation is utilized in the bolt on Cellulose grind. The assay of starch in the fiber feed product is reduced to less than 0.02%. When the cellulose grind is not added, the balance of the fractionation is illustrated in FIG. 3 Basic Bio Grind. The LAI Bio Grind with Fermenter and Cellulose Feedstock is illustrated in Figure Four.

9. Fractionation and Blending

The overriding premise for sustainability of this ICBR or any of the individual or of plurality Corn Dry, Wet, Bio or Cellulose Grinds for no waste of product, nutritional value, quality or water weight “Water Weight” which increases the shipping costs and reduces the value. Water weight is anything that is wasted from processing to consumption of the feed by the animal including over drying creating negative water weight by replacing moisture with product and products that merely dilute the value product or reduces the density which increases the shipping cost. The major problem is failure to blend to meet the nutritional requirements of the animal thus having no nutritional value such as water or utilizing volume without nutrition to the animal consuming the product. Priority is given to fractionation of protein to produce Corn Gluten Meal which can be produced from Gluten Protein, available Corn Steep Solids, available Yeast Cream, reclaimed fermentation catalyst or available Corn Oil Meal either wet for nearby pigs or chickens feeders. The second priority would be WDGS for nearby ruminants and DDGS for drying and long shipping or export. These products can be supplied at protein levels of Corn Gluten Feed. Low Oil DGS/standard protein, or Low Oil/high protein as desired by the client.

Any available waters or cellulose biomass products can be recycled for reprocessing with the Cellulose Grind processes. A complete presentation of the steps, products, co-products and recycles are presented in FIG. 11 ICBR GRIND-LAI

Integrated dry, wet, bio and cellulose grinds as a complete grind at one location or multiple locations with finished co-product refining at selected locations for economics of collecting feedstock, shipping backhauls between plants, available facilities at the locations including available of interstates, railroads and barge terminals for incoming feedstock and shipping especially for export.

10. Economic Advantages

One major advantage of the ICBR process is that it is a sustainable answer to the depressed prices of the DDGS by-product of the dry grind ethanol process. After the value added protein Co-Products of the Bio Grind are fractionated the remaining corn fiber becomes a conditioned feedstock for the cellulose grind at half the cost of collecting and conditioning biomass feedstock. In addition, when the recycle water, heat and catalyst becomes balanced, the available cellulose provide half of the feedstock to increase the yield from Cellulose Processing by 25% to an equivalent of 4 gallons per bushel corn feedstock for Fuel Ethanol with no process product loss including the Food Wet Grind which stand alone typically provides only 2.75 gallons per bushel feedstock.

By providing stand alone fermentation with continuous pervaporation fractionation of ethanol to the primary continuous fermenter/distillation/anhydrous blending system the becomes considerable reduction of equipment, costs, energy and chemicals. Special sugars can be can be treated with special conditions to reduce the costs of specialty enzymes. The LAI Bio Grind bolt on Cellulose Process ICBR Integrated Grind Pervaporator Process is outlined in Figure Ten. This invention provides the technology to be the lowest cost cellulose ethanol in the industry. There will be no product waste from the Cellulose Grind. Any product that cannot be recycled or used for animal Value Co-Products including lignin will be gasified or concentrated for boiler feedstock.

The United States is the largest corn producing country in the world. The Corn Wet Grind industry represents the largest source of refined starch for both food and non food applications. With the increased demand for ethanol as a fuel additive, a low cost process has evolved and resulted in the Dry Grind Ethanol process for increased yields of ethanol at the expense of eliminating food applications and wasting nutritional value of animal feed co-products. Dry Grinds have a single bi-product of Distiller Grains and Solubles. W/D DGS can replace corn for animal feed rations but is limited for ruminants due to excess oil and other animals due to high fiber and low proteins. Quality is depressed due to the product being subjected to high temperature and harsh chemical reactions.

As the industry expands three (3) problems become apparent and one solution becomes evident: 1. Corn fed to animals or used for the Dry Grind Processes reduce the availability of food. 2. Increased volumes of DGS depress the domestic market value below the nutritional corn related value of 1.4× the value of corn as the industry expanded in the past 10 years to a low of one half times. 3. Added handling and transportation costs due to the added weight and the reduced density of oils and fibers depress the value of the protein that is in high demand for non growing areas and export countries. This invention outlines integration of Dry, Wet, Bio and Cellulose Grinds for the best yields of value products for improved profitable net-corn-costs.

There are four major breakthrough inventions that readily fit as replacement continuous processes that can retrofit into all of the Dry, Wet, Bio and Cellulose Grinds that allow the grinds to be integrated into one continuous Integrated Corn Based Refining (ICBR) process. These include: 1. LAI Continuous Steeping that can be completely automated to handle a wide range of inlet moisture, scalped or cleaned corn, with or without sulfur dioxide, non aerated circulation for anaerobic steeping, self-cleaning screens and feed nozzles, in place corn washing, membrane conditions for shorter water up-take times with a smaller foot print and operating cost. (Figure Five—ICBR Wet Grind—Food Grade Steep). 2. LAI Supramyl Liquefaction with combined fine grinding to shatter the starch granule for improved shear in one process step with low pressure recycle heat, reduced temperature, higher solids, less enzyme and chemicals, with reduced viscosity and 3-5% better sugar yields for improved ethanol yields. (Figure Six—ICBR Bio Grind Liquefaction). 3. LAI Continuous Fermentation that utilizes carbon dioxide Fine Gas Bubble supplemental mixing combined with tank blanketing to control anaerobic processing to control aerobic bacteria contaminant growth and ethanol yield losses to allow yeast and catalyst recycle and reduce need for bacteriostatic agents. (Figure Seven—ICBR Bio Grind anaerobic Fermentation). and 4. Membrane cross-flow filter ethanol fractionation by pervaporation to concentrate dilute ethanol produced with the Cellulose Grind One Stage Continuous fermenters to reduce distillation and evaporation equipment and steam. (Figure Ten—ICBR Cellulose Grind Pervaporator Process).

Along with the breakthrough fractionation patented inventions of LAI Bio Grind corn and fiber refining, recent technology advances in enzymes, yeasts, contaminant control and membrane technology can be fully implemented to streamline the process and re-use the recycled water as needed. Nutritional value of the corn that is reduced as much as 30% with existing Dry Grind processes can be retained and be used for human food and animal feed. The plant will have a sustainable process that will not only have no waste but will be able to process waste from other processes. The plant will produce feedstock for bolt-on corn bio based processes.

Why is sustainability important? There can be no waste. To continue operating at a profit, it must be more efficient. It must operate more efficiently in its use resources especially of its use of energy. The largest use of energy is liquefaction in industry today. The Supramyl Process uses mechanical energy with supplemental low pressure re-used steam, lower temperatures and less dilution to control viscosity. The entire process operates with considerable recycle, higher dry substance which ends up in less water added, less need evaporation and fewer gallons feed to the distillation unit. This is what it is going to take to make ethanol plants competitive and allow one again to take their place as the most productive Agri-Processing establishments for Food, Feed, Fiber and Fuel from renewable sources Corn and Biomass. The LAI-ICBR very well can be sustainable by becoming the lowest cost in the sector. The plant must also be Safe, Clean, Compliant and Closed loop with no Product waste with fractionation priority refining to the highest value products with the highest demands that meet or exceed corn related prices to support improvement in the net-corn cost. Fuel Ethanol Prices are controlled by demand and Crude oil prices and can be at break even or below corn prices. The integration of the four current Grind Processes offers added value product potential for a sustainable profit margin net-corn-cost.

Dry Grind plants of all sizes can be retrofitted with the ICBR continuous steep for Steep Syrup and food germ fractionation. A central location can be located to fractionate Corn Oil Meal and Corn oil which can be refined. Food Grade starch can be fractionated to add feedstock for production of value added starch, protein and fiber based bio processing with the larger uses at select locations. All waters and byproducts are blended with the DRY Grind and then processed with the LAI BIO GRIND and added CELLULOSE GRIND to finish quality feeds with no nutritional losses and 4 or more gallon of fuel ethanol per feedstock corn equivalent starch and sugar.

A single plant with a minimum of 300,000 to 500,000 bushel per day or a coop within a reasonable driving distance can retrofit with a LAI-ICBR to optimize processes for a sustainable net-corn-cost as corn costs fluctuate.

11. Technical Advantages

The process of this invention has many technical advantages over prior art processes. Some of the advantages are illustrated in Figures Thirteen and Fourteen, other advantages include the following.

The process can handle high moisture feedstock for using early harvesting without cost of drying for processing or storage.

The process features continuous processing including steeping with or without sulfur dioxide and de-aerated steep water for anaerobic steeping to eliminate wild yeast and contaminant bacterial growth to improve starch and ethanol yields.

The process features a reduced plant footprint and required equipment with the reduced requirement for steeping, food grade fractionation, liquefaction, saccharification, fermentation, pervaporation, distillation, evaporation, dewatering and finishing Fuel Ethanol.

The process improves feedstock cost with the facilities and processes for adding lower cost starch adjuncts and utilizing washed fiber at half the cost for cellulose feedstock.

Improving starch and ethanol yield from Wet Grind 2.75G/Ebu, Dry Grind 2.80G/EBu, Bio Grind 2.85G/Ebu to the LAI ICBR Grind 3.0G/Ebu with and additional 25% attributed to the Bolt on Cellulose Grind for 4.0 G/Ebu at the lowest cost Fuel Ethanol in the industry and the best net-corn-cost due to the added Value Co-Products at corn related or better prices.

Quality products for no loss of nutrition or “water weight” during processing and no potential waste of food products in lieu of use for Fuel Ethanol production.

Reduction of need for evaporation due to Bio Grind processing at 42% dry substance and low viscosity in lieu of 36% when not using the LAI Supramyl liquefaction/fine grind process.

Continuous fermentation with anaerobic processing provided with fine bubble Carbon Dioxide supplemental mixing and blanketing for the reduction of contaminants for reuse of the catalysts in secondary fermenters.

The advantages can be adapted to continuous or batch operations.

The process can be installed one or more steps at a time both at minimum pilot rates of 2,000 to 4,000 bushels per day and minimum commercial rates of 40,000 to 60,000 bushels per day with no upper limit.

There are seven steps that can be installed one or more at a time.

The pilot steps can be fabricated on a skid and shipped anywhere in the world, operated individually, or tied into existing operations.

The larger plant rates are the most economical as the process provides ample feedstock for refining rates economical for producing all food products identified.

I claim:

1. A sustainable integrated bio grind refining process for a feedstock of corn and corn stover, the process operating batch or continuous to produce fuel ethanol, the process comprising:
(a) feeding 15 to 30 percent of the feedstock to a wet grind stream;
(b) feeding 15 to 18 percent of the feedstock to a dry grind stream;
(c) feeding 15 to 30 percent of the feedstock to a ligno cellulose stream; and
(d) processing the feedstock to form germ, steep liquor, high protein soft starch, pre-liquefaction corn germ, post-distillation corn oil, and by-products of standard DPGS, low oil DPGS, high protein DPGS, and batch feed lignin.

2. A process for refining a feedstock comprising corn starch containing materials and plant bio-mass containing cellulose that can be converted with a process for food priority, animal feed, fuel ethanol, and no process loss, the process comprising:
(a) providing corn kernels having a moisture content of 10 to 30 percent and comprising: (i) a pericarp comprising coarse fiber; (ii) an endosperm comprising soft starch, hard starch, protein, and fiber; and (iii) a germ;
(b) screening the feedstock to remove 20 to 40 percent of the corn to remove small grain, broken grain, and elevator dust, and then diverting to a secondary process for conventional dry grind processing;
(c) steeping the corn kernels in recycled water from down stream processes, which water has a temperature of 125 to 160° F., is essentially free of sulfurous acid and contains effective amounts of amylase enzymes in a counter-current steeping reactor system for about 10 to 20 hours to produce an aqueous slurry of steeped corn kernels having a moisture content of about 40 to 50 percent;
(d) washing the steeped corn and fractionate the steep liquor to be used as fermentation media, blending protein, and animal feed;
(e) coarsely grinding the steeped corn kernels to produce a coarsely ground stream comprising coarse fiber, soft starch, hard starch-protein-fine fiber fragments and germs;
(f) reducing the dry substance to 17 to 18 percent to hydro-float fractionate the germ and any other starch product to be used for food and transferring the balance to a Supraton System for blending to 40 to 42 percent DSS for feedstock;
(g) blending the dry grind feedstock to drop the moisture to 15 to 18 percent moisture, and replacing the corn used for food in the bio/wet grind stream with the volume planned for the dry milling stream;
(h) cleaning the corn and adding the corn cleanings-off-all to the Supraton System for DSS blending;
(i) coarse grinding the cleaned dry grind food products required and sending the balance to the Supraton System for blending and controlling the feedstock to raise the typical dry grind fermented dry substance from 34 to 36 to 40 to 42 dry substance starch;
(j) subjecting the partially gelatinized stream to sufficient shear and cavitation forces in the presence of effective amounts of amylase enzymes to gelatinize substantially all the starch and to produce a liquefied stream at a viscosity typicaly of the dry grind 34 to 36 DSS level;
(k) exposing the liquefied stream to effective amounts of amylase enzymes to produce a saccharified stream reducing the viscosity for fractionating the remaining insoluble corn grain to produce a clean soluble stream to the fermenter;
(l) adding an effective amount of yeast to the saccharified stream and fermenting it to produce carbon dioxide and a fermented stream containing ethanol;
(m) scrubbing the carbon dioxide removed from the fermenter to recover retained ethanol;
(n) returning sufficient carbon dioxide for the agitation assist fine bubble system for anaerobic fermentation and head space blanketing; and
(o) finishing the refining of the ethanol in the distillation system.

3. A sustainable process for producing ethanol from corn, cereal grain, adjuncts of cereal grain processing, corn stover, adjuncts of biomass material, starch containing tubers, or starch containing adjuncts of rejects or re-process materials, the process comprising:
(a) Corn Wet Grind with food fractionation with continuous steeping;
(b) Corn Dry Grind with cereal grin adjunct and counter flow bi-products and wash water blending;
(c) Corn Bio Grind with LAI Supramyl high solids feed, high speed centrifugal colloid mill, combination fine grind/Liquefaction with high shear and low viscosity discharge. The discharge is separated for addition to the combined fermentation system and the animal feed refining system with the balance to feed the Cellulose Grind;
(d) The cellulose grind utilizes available fiber, heat, water, catalyst, chemicals, enzyme and yeast recycles from the Bio Grind, adjunct from the Dry Grind and additional feedstock material containing cellulose, hemicellulose, lignin, and pectin;
(e) The combined fermentation process includes three continuous fermenters comprising: (i) The Bio Grind LAI continuous cascade fermenter process with Carbon Dioxide fine bubble supplemental mixing and blanketing for anaerobic yeast ethanol production; (ii) the invention of a one stage continuous Pentose utilizing recycled catalyst and pervaporation filter fractionation of ethanol discharged to the primary fermenter for concentration; and (iii) the invention of a one stage fermenter for the discharge of the cellulose/dextrose hydrolysate process. The evaporation and animal feed product is combined with the Bio Grind fermentation and animal feed processes; and
(f) The animal feed fraction is washed to the fermenters. The fiber, Corn steep syrup and protein are blended with a priority to produce the corn value products with the best demand and price.

4. The process of claim 3 wherein the wet grind food fractionation continuous process comprises:
(a) Providing corn kernels having a moisture content of about 10 to 30 percent moisture comprising: (i) a pericorp comprising of coarse fiber; (ii) an endosperm comprising of soft starch, hard starch, protein and fine fiber; and (iii) a germ;
(b) Steeping the kernels in recycled deaerated water counter current food grade processes with or without Sulfur Dioxide utilizing the patented LAI Continuous steeping process with anaerobic conditions for control of fermentation contamination, contaminant control and conditions for water uptake for membrane conditions to reduce steeping time;
(c) The continuous steep overflow fraction is a quality corn steep solids (CSS) for fine bio fermentation feedstock catalyst or concentrated for marketing, the internal washing of the steeped corn provides the feedstock for the fractionation of the germ;
(d) The steeped corn at 44-45% moisture is course ground to free the germ from the kernel and diluted to 16-18% moisture with recycled downstream water and fractionated from the heavier mash with a hydrocyclone process, the germ overflow can be conditioned and refined to crude, degummed or refined food grade oils and quality Corn Oil Meal, the underflow is fine ground and used for feedstock for production of Bio chemicals and products or quality feedstocks for fermentation products or fractionated for food grade starch and other starch products;
(e) The balance of the underflow is fractionated as needed for first strike fractionation of washed starch for food and treated starch, washed starch for liquefaction to sugar food products, washed gluten for food and process products and washed fiber for dietary food product and other processed products; and
(f) The bi-products of the fractionation processes, the wash water is recycled to the Dry Grind Process along with product not used for fractionation.

5. The process of claim 3 wherein the Dry Grind Process comprises:
(a) Providing dent corn or other cereal grains such as milo and adjunct of other starch containing grain products of 10-18% moisture for course grinding; and
(b) Add all available feedstock, bi-products and wash water available from the wet grind fractionation and other adjuncts of dextrose and starch liquid adjuncts, the blend is adjusted to 40-42% for the feedstock to Bio Grind Process, downstream waters are used in lieu of fresh water.

6. The process of claim 3 wherein the Bio Grind Process comprises:
(a) Providing soaked course ground corn and or starch containing grain starch product and fractionate balance from the food grade wet grind step and liquid reprocess sugars at depressed prices or competitive prices and concentrate;
(b) Preheat the 40-42 DS feedstock and fine grind and liquefy with the LAI Supramyl patented Liquefaction process;
(c) Fractionate the fiber and protein for animal feed blending with distillers solubles and washed fiber balance for cellulosic ethanol feedstock; and
(d) Sugars are blended with steep water and yeast for fermentation feedstock.

7. The process of claim 3 wherein the LAI Cellulose Grind process for refining corn fiber, corn stover and/or adjunct plant materials comprises:
(a) Providing a plant material comprising: (i) starch; (ii) sugar; (iii) fiber comprising cellulose, hemicellulose, lignin and pectin;
(b) Heating the fiber slurry under mild alkaline conditions to hydrolyze substantially all of the hemicellulose and pectin into soluble pentose and hexose sugars and treating with polygalacturonase as needed;
(c) Separating the insoluble cellulose and lignin from the pentose and dextrose sugars fermenting for ethanol with dextrose and pentose yeasts;
(d) Fractionate the ethanol with pervaporation filtration and return for concentration, distillation, dehydration and fuel blending in the Ethanol Bio Grind process;
(e) Heating the slurry of cellulose and lignin under severe alkaline conditions to hydrolyze substantially all of the lignin into soluble hydrolysate for fractionation for boiler feed stocks and enzyme treating; and
(f) Treating the resulting insoluble cellulose with an effective amount of cellulose enzyme to convert to a secondary glucose solution and adding the glucose to the secondary yeast fermenter.

8. The process of claim 3 wherein the fermentation and ethanol refining for both starch and cellulose ethanol processes will be combined for finishing comprising:
(a) A one stage full mash pentose fermenter with special enzymes and yeast to ferment xylose, arrabinose and other DP2 and higher sugars available in the recycle catalyst and other feedstock, the ethanol will be partially concentrated with the pervaporation membrane filter system, the permeate filtrate will be concentrated in the final continuous cascade anaerobic process, the concentrate syrup solids will be evaporated for production of animal feed with the ethanol in the condensate reclaimed in the pervaporation membrane filter system;
(b) A second one stage full mash fermenter with special enzymes and yeast will be used to ferment the product of high temperature cellulose hydrolysis, the ethanol will be partially concentrated with the pervaporation membrane filter system, the permeate filtrate will also be concentrated in the final continuous anaerobic process, the concentrate syrup solids will be evaporated for production of animal feed with the ethanol in the condensate reclaimed in the pervaporation membrane filter system; and
(c) The final continuous cascade anaerobic fermenter process with partial fine bubble mixing and blanketing with carbon dioxide will be used to ferment and concentrate fermenter ethanol for refining Fuel Ethanol.

9. The process of claim 3 wherein animal feed is refined by blending Bio Grind pre-fermentation fractionated protein and fiber from post Cellulose Grind and post distillation insoluble product comprising:
(a) Fractionation product available from Pre-Fermentation for Corn Gluten Meal, DGS, Corn Gluten Feed with balance becoming feedstock for the Cellulose grind;
(b) Balance of product from fractionation of ethanol from Cellulose Ethanol fermentation is blended with the Bio Grind product with any remaining partially hydrolyzed cellulose returned to the Cellulose Grind;
(c) Soluble solids available from the steam distillation are returned to pre-fermentation blending;
(d) Waters containing soluble process solids are recycled back to the Dry Grind for washing, wet scrubbing environmental discharges, or dilution; and
(e) There are no process solids waste discharges.

10. The process of claim 3 in which quality is improved and no nutritional value of corn is lost due to harsh temperature and chemicals are used.

11. The process of claim 3 in which Food fractionation and refinery has priority and wash water is concurrent recycled to processing of Fuel Ethanol and Animal feed products.

12. The process of claim 3 in which starch liquefied before fractionation providing a Fuel ethanol from starch to 3.0 gallon per corn bushel equivalent and Recycled catalyst for cellulosic ethanol an additional 1.0 gallon per equivalent corn feedstock, producing a 5% improvement over most Dry Grind plants, 8.5% improvement over most Wet mill Food Refineries and 15% over cellulose refineries with no additional catalyst.

13. The process of claim 3 wherein Wash water used for Wet Grind Food Refining is transferred counter current for processing with no waste.

14. The process of claim 3 in which the steeping utilizes 10-30% moisture corn for feedstock and requires no added sulfur dioxide, the steep operates anaerobically with no steeping fermentation loss and is designed for optimum conditions for membrane flux to optimize steeping rates and soluble fractionation yields.

15. The process of claim 3 in which food grade germ is fractionated at 50% oil.

16. The process of claim 3 in which the mash after germ fractionation is partially ground to leave corn grits in lieu of fine fiber which is transferred to the Dry Grind process for blending and liquefaction before fractionating to produce fine fiber of less than 0.2% starch to feed the Cellulose system and wherein first cut is only is used for low cost fractionation and refining of food products.

17. The process of claim 3 in which distressed corn and other grains and grain products are used to supplement food grade fractionated products for ethanol liquefaction for reduced cost for solids control and starch liquefaction for Fuel Ethanol.

18. The process of claim 3 comprising the use of recycle heat and low pressure steam (5 Bar, no flashing, no vapor), low temperature (200-220 F), reduced steam and enzyme and less evaporation due to processing at 15% higher than typical jet systems, less retrogradation, color and by-products (Maillard Reaction) and viscosity reduced internally for low viscosity discharge for ease of fractionation.

19. The process claim 3 in which the pervaporation process invention significantly reduces the size of the distillation and evaporation steps due to reduced volume and steam for distillation.

20. The process of claim 3 where all of the by-product are used for quality animal feed or recycled or for re-processing.
